(19) United States
(12) Patent Application Publication
(10) Pub. No.: US 2024/0252090 A1
(43) Pub. Date: Aug. 1, 2024

(54) SYSTEM FOR DETERMINING CARDIOVASCULAR CHARACTERISTICS

(71) Applicants: Tiffany WU, New York, NY (US); CHI-HUA FOUNDATION, New Taipei City (TW)

(72) Inventors: Chau-Chung WU, New Taipei City (TW); Meng-Tsung LO, New Taipei City (TW); Wei-Yu CHEN, New Taipei City (TW); Zeus HARNOD, New Taipei City (TW); Tiffany WU, New York, NY (US)

(73) Assignees: Tiffany WU, New York, NY (US); CHI-HUA FOUNDATION, New Taipei City (TW)

(21) Appl. No.: 18/563,669

(22) PCT Filed: May 17, 2022
(86) PCT No.: PCT/US2022/029575

§ 371 Date: Nov. 22, 2023

(30) Foreign Application Priority Data
May 27, 2021 (TW) 110119296

(51) Int. Cl. A61B 5/332; A61B 5/00; A61B 5/256; A61B 5/282; A61B 5/352; A61B 5/353; A61B 5/355; A61B 5/358; A61B 5/36

(52) U.S. Cl. CPC A61B 5/332; A61B 5/256; A61B 5/282; A61B 5/352; A61B 5/353; A61B 5/355; A61B 5/358; A61B 5/36; A61B 5/6805

(57) ABSTRACT

A system for determining cardiovascular characteristics is to be disposed on the body of a subject. The body has a detection area. The system includes a detector member and a processor. The detector member includes at least sixteen precordial electrodes which are to be placed on the chest of the subject within the detection area and which produce at least sixteen electrocardiogram (ECG) signals. The processor calculates at least twenty-four characteristic values based on the ECG signals. The characteristic values serve as basis for determining a location of chronic or acute myocardial ischemia in the body and a region of chronic or acute myocardial ischemia in the heart of the subject.

### FIELD

The disclosure relates to a medical examination system, and more particularly to a system for determining cardiovascular characteristics.

### BACKGROUND

The medical guidelines recommend that a patient having a myocardial infarction should receive cardiac catheterization no more than 90 minutes after the patient's arrival at an emergency department. Coronary arteries that transport oxygenated blood to the heart muscles include the right coronary artery (RCA), the left anterior descending artery (LAD) and the left circumflex artery (LCX). Depending on which one of the coronary arteries is blocked, a corresponding intervention procedure should be adopted. Therefore, how to evaluate whether a patient has myocardial ischemia and how to determine the location of myocardial ischemia in a short period of time are crucial.

A conventional method for evaluating the location of myocardial ischemia is implemented by referring to a 12-lead electrocardiogram (ECG) of a patient. A medical professional needs to perform comprehensive evaluation based on ECG waveforms reflecting ST segment elevation or depression in different grouped leads, such as the precordial leads V₁-V₆, the inferior leads II, III and aVF, and the lateral leads I, aVL, V₅ and V₆.

However, since elevation or depression in the waveform of ST segment is often unnoticeable in the early stage of myocardial ischemia, in practice, it is difficult to complete the evaluation in a short period of time.

For a patient with chronic myocardial ischemia, since signs of ischemia may be less noticeable, the diagnostic sensitivity is relatively lower when reference is made to a resting ECG which is recorded while the patient is at rest. Therefore, a stress ECG may be recorded while the patient is exercising or is given an inotrope to induce myocardial ischemia in the stress ECG test for evaluating the possibility of coronary artery disease. However, this approach is not applicable to patients who are unsuitable for exercise (for example, the elders or people with reduced mobility). Moreover, the whole procedure of stress ECG is complicated, with some risk and time-consuming.

In addition, for both resting and stress ECG, the waveform of ST segment may be easily influenced by chest wall impedance, noise and baseline shift, which would result in evaluation error. Even though there is a conventional system for evaluating the cardiovascular condition of a subject with hundreds of electrodes to obtain spatial ECG signals in an attempt to promote the detection accuracy, there is an issue of high cost. Moreover, it is difficult to keep such large number of electrodes simultaneously attached to the body of a patient.

### SUMMARY

Therefore, an object of the disclosure is to provide for determining cardiovascular a system characteristics that can alleviate at least one of the drawbacks of the prior art.

The system for determining cardiovascular characteristics is to be disposed on the body of a subject. The body has a detection area which is defined by a right edge of the sternum, a horizontal line passing through the first intercostal space, the left midaxillary line, and a horizontal line passing through the eighth rib of the body. The system includes a detector member, a processor and an output unit.

The detector member includes four limb electrodes to be placed on limbs of the subject, and at least sixteen of precordial electrodes to be placed on the chest of the subject and spaced apart from each other. The limb electrodes and the precordial electrodes respectively measure the electrical potentials at locations of the respective limb and precordial electrodes, and cooperatively produce at least sixteen electrocardiogram (ECG) signals. Each of the ECG signals includes the P, Q, R, S and T waves. The precordial electrodes are to be placed within the detection area in a manner that
at least two of the precordial electrodes are placed at locations corresponding to the right edge of the sternum, at least three of the precordial electrodes are placed at locations corresponding to a left edge of the sternum, at least three of the precordial electrodes are placed at locations corresponding to a middle line which is midway between the left edge of the sternum and the left midclavicular line of the body, at least four of the precordial electrodes are placed at locations corresponding to the left midclavicular line, at least two of the precordial electrodes are placed at locations corresponding to the left anterior axillary line of the body, and at least two of the precordial electrodes are placed at locations corresponding to the left midaxillary line of the body, and
at least three of the precordial electrodes are placed at locations corresponding to the third intercostal space of the body, at least five of the precordial electrodes are placed at locations corresponding to the fourth intercostal space of the body, at least four of the precordial electrodes are placed at locations corresponding to the fifth intercostal space of the body, at least one of the precordial electrodes is placed at locations corresponding to the sixth intercostal space of the body, and at least three of the precordial electrodes are placed at locations corresponding to the middle line which is midway between the left edge of the sternum and the left midclavicular line of the body, and within a range from the third intercostal space to the sixth rib of the body.

The processor is in signal communication with the detector member to receive the ECG signals. The processor is configured to calculate at least twenty-four characteristic values based on the ECG signals, wherein the at least twenty-four characteristic values are respectively dedicated to at least twenty-four characteristic locations on the chest of the subject within the detection area. The characteristic locations include the locations of placement of the precordial electrodes. The characteristic values serve as bases for determining a location of chronic or acute myocardial ischemia in the body and a region of chronic or acute myocardial ischemia in the heart of the subject.

The output unit is electrically connected to the processor and is controllable by the processor to output the characteristic values.

### BRIEF DESCRIPTION OF THE DRAWINGS

Other features and advantages of the disclosure will become apparent in the following detailed description of the embodiment (s) with reference to the accompanying drawings, of which:

FIG. 1 is a block diagram illustrating an embodiment of a system for determining cardiovascular characteristics according to the disclosure;

FIG. 2 is a schematic diagram illustrating a detection area on the chest of the body of a subject;

FIG. 3 is a schematic diagram illustrating an example of locations of placement of sixteen precordial electrodes on the chest;

FIG. 4 is a schematic diagram illustrating an example of a comparison chart according to the disclosure;

FIG. 5 is a flow chart illustrating an embodiment of a method to determine the region and severity of myocardial ischemia according to the disclosure;

FIG. 6 is an example of a color map for presenting characteristic values with respect to a first exemplary case where sixteen ECG signals were obtained from a subject suffering from left circumflex artery (LCX) stenosis;

FIG. 7 is an example of a color map for presenting characteristic values with respect to a second exemplary case where sixteen ECG signals were obtained from a subject suffering from right coronary artery (RCA) stenosis;

FIG. 8 is an example of a color map for presenting characteristic values with respect to a third exemplary case where sixteen ECG signals were obtained from a subject suffering from left anterior descending artery (LAD) stenosis;

FIG. 9 is an example of a color map for presenting characteristic values with respect to a fourth exemplary case where sixteen ECG signals were obtained from a subject suffering from three-vessel disease (3VD);

FIG. 10 is similar to FIG. 3 and illustrates an example of locations of placement of twenty-four precordial electrodes on the chest;

FIG. 11 is similar to FIG. 6, and is an example of a color map for presenting characteristic values with respect to the first exemplary case but with twenty-four ECG signals being obtained;

FIG. 12 is similar to FIG. 7, and is an example of a color map for presenting characteristic values with respect to the second exemplary case but with twenty-four ECG signals being obtained;

FIG. 13 is similar to FIG. 8, and is an example of a color map for presenting characteristic values with respect to the third exemplary case but with twenty-four ECG signals being obtained;

FIG. 14 is similar to FIG. 9, and is an example of a color map for presenting characteristic values with respect to the fourth exemplary case but with twenty-four ECG signals being obtained;

FIG. 15 is similar to FIG. 3 and illustrates another example of locations of placement of twenty-four precordial electrodes on the chest; and

FIG. 16 is similar to FIG. 3 and illustrates an example of locations of placement of thirty-six precordial electrodes on the chest.

### DETAILED DESCRIPTION

Before the disclosure is described in more detail, it should be noted that where considered appropriate, reference numerals or terminal portions of reference numerals have been repeated among the figures to indicate corresponding or analogous elements, which may optionally have similar characteristics.

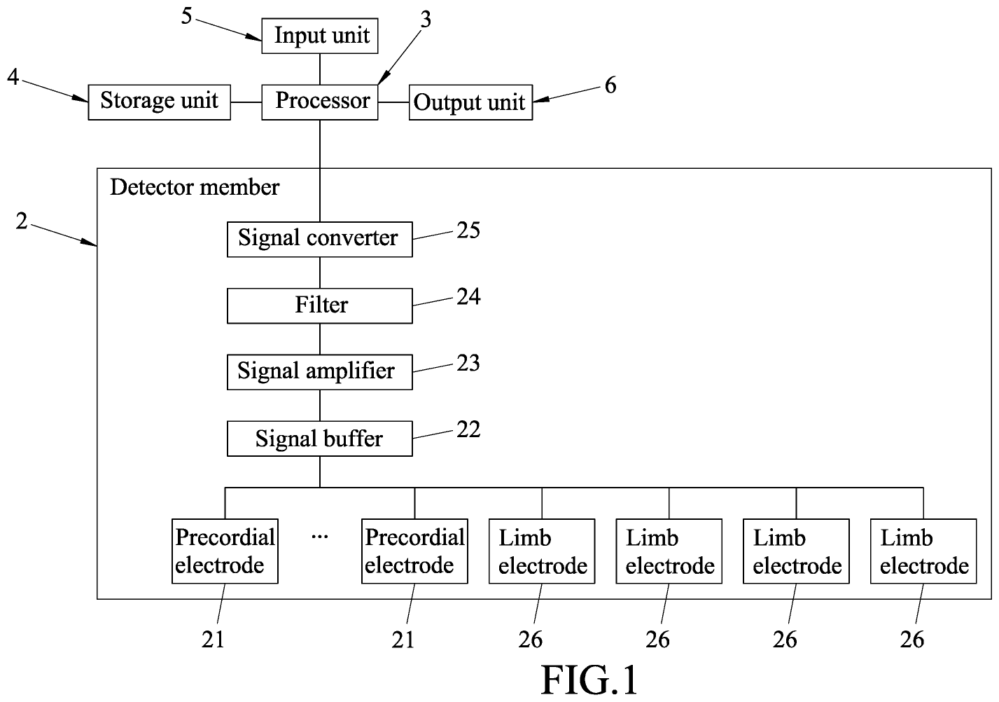

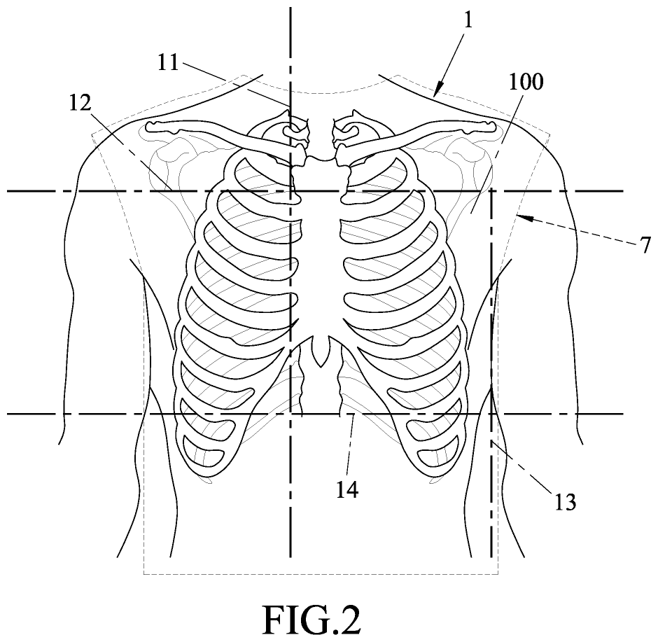

Referring to FIGS. 1 and 2, an embodiment of a system for determining cardiovascular characteristics according to this disclosure is adapted to be used on a body 1 of a subject, such as a patient under examination. The body 1 has a detection area 100 on the chest thereof. The detection area 100 is defined by a right edge 11 of the sternum, a horizontal line 12 passing through the first intercostal space, the left midaxillary line 13 and a horizontal line 14 passing through the eighth rib of the body 1.

The system includes a detector member 2, a processor 3, a storage unit 4, an input unit 5, an output unit 6, and a wearable unit 7. The processor 3 is in signal communication with the detector member 2, the storage unit 4, the input unit 5 and the output unit 6.

The input unit 5 may be any type of input device that is able to be operated for inputting a command to the device, such as, but not limited to, a voice input device, a video input device, a touchscreen, a keyboard or a pointing device.

The detector member 2 includes four limb electrodes 26 to be placed on limbs of the subject and a plurality of precordial electrodes 21 to be placed on the chest of the body 1 in a manner that the precordial electrodes 21 are spaced apart from each other and within the detection area 100. At least some of the precordial electrodes 21 are to be placed on the left chest of the body 1 of the subject. The processor 3 is configured to control, in response to receipt of an activating command inputted via the input unit 5, the electrodes 26, 21 to respectively measure electrical potentials at their respective locations of placement, and to cooperatively produce a plurality of electrocardiogram (ECG) signals. Each of the ECG signals includes the P, Q, R, S and T waves. The combination of the Q, R and S waves is referred to as the QRS complex.

In some embodiments, to produce each of the ECG signals, the electrical potential measured by a respective one of the precordial electrodes 21 is used as a positive pole, one, or a combination of two or more of the electrical potentials measured by the limb electrodes 26 is used as a negative pole, and the electrical potential difference between the positive pole and the negative pole is detected to produce the ECG signal. In this way, the ECG signals respectively correspond to the precordial electrodes 21, and thus respectively correspond to the locations of placement of the precordial electrodes 21. In some embodiments, a number of the precordial electrodes 21 is sixteen or more.

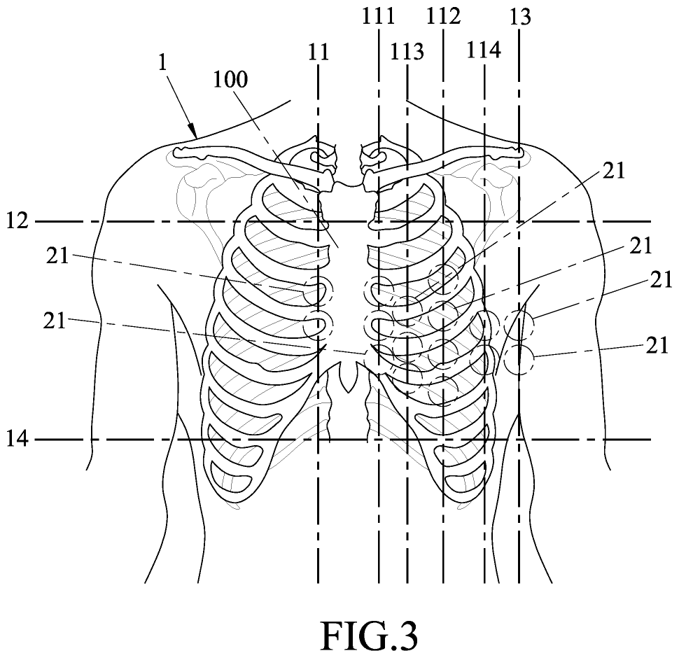

Referring to FIG. 3, with respect to locations of placement of the precordial electrodes 21 from middle to side along the lateral direction of the body 1, at least two precordial electrodes 21 are placed at locations corresponding to the right edge 11 of the sternum, at least three are placed at locations corresponding to a left edge 111 of the sternum, at least three are placed at locations corresponding to a middle line 113 which is midway between the left edge 111 of the sternum and the left midclavicular line 112 of the body 1, at least four are placed at locations corresponding to the left midclavicular line 112, at least two are placed at locations corresponding to the left anterior axillary line 114 of the body 1, and at least two are placed at locations corresponding to the left midaxillary line 13 of the body 1.

With respect to locations of placement of the precordial electrodes 21 from top to bottom along the inferior direction of the body 1, at least three precordial electrodes 21 are placed at locations corresponding to the third intercostal space of the body 1, at least five are at locations corresponding to the fourth intercostal space of the body 1, at least four are at locations corresponding to the fifth intercostal space of the body 1, at least one are at a location corresponding to the sixth intercostal space of the body 1, and at least three are placed over the middle line 113 which is midway between the left edge 111 of the sternum and the left midclavicular line 112, and at locations within a range from the third intercostal space to the sixth rib of the body 1.

Referring to FIG. 3, in this embodiment, the number of the precordial electrodes 21 is sixteen. From middle to side along the lateral direction of the body 1, two precordial electrodes 21 are placed at locations corresponding to the right edge 11 of the sternum, three at locations corresponding to the left edge 111 of the sternum, three at locations corresponding to the middle line 113 which is midway between the left edge 111 of the sternum and the left midclavicular line 112, four at locations corresponding to the left midclavicular line 112, two at locations corresponding to the left anterior axillary line 114, and two at locations corresponding to the left midaxillary line 13. From top to bottom along the inferior direction of the body 1, three precordial electrodes 21 are placed at locations corresponding to the third intercostal space, five at locations corresponding to the fourth intercostal space, four at locations corresponding to the fifth intercostal space, one at locations corresponding to the sixth intercostal space. In addition, three precordial electrodes 21 are placed at locations each corresponding to an intersection of the middle line 113 with a respective one of the fourth rib, the fifth rib and the sixth rib of the body 1.

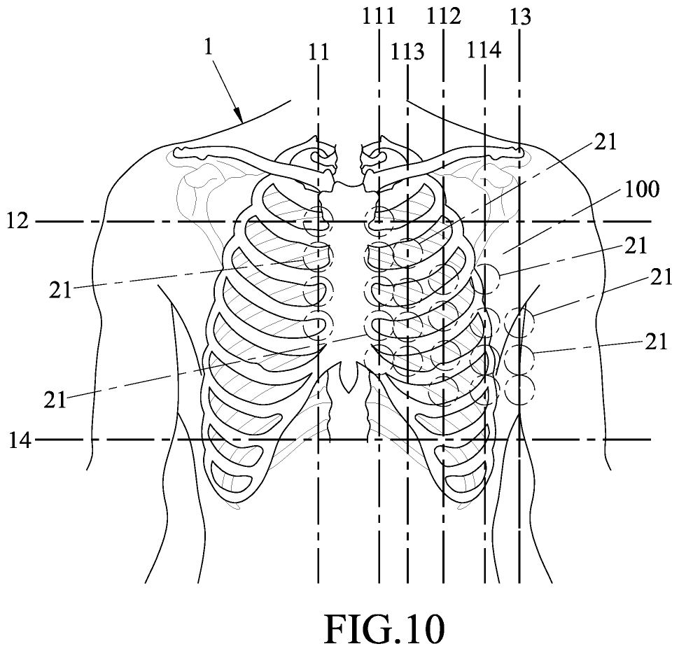

In order to clearly illustrate the arrangement of the wearable unit 7 and the precordial electrodes 21 on the body 1, the wearable unit 7 is depicted by broken lines in FIG. 2, and the precordial electrodes 21 are depicted by two-dash lines in FIG. 3 and subsequent FIGS. 10, 15 and 16.

The detector member 2 further includes a signal buffer 22 electrically connected to the electrodes 26, 21, a signal amplifier 23 electrically connected to the signal buffer 22, a filter 24 electrically connected to the signal amplifier 23, and a signal converter 25 electrically connected to the filter 24. The signal buffer 22 provides a sufficiently large input impedance for coupling the ECG signals produced by the electrodes 26, 21 to the signal amplifier 23. The signal amplifier 23 amplifies the ECG signals, and transmits the ECG signals thus amplified to the filter 24. The filter 24 filters out noise in the ECG signals and interference accompanying a power source signal provided to the system. The signal converter 25 converts the ECG signals which have passed through the filter 24 to digital form, and transmits the ECG signal thus converted to digital form to the processor 3 for analysis.

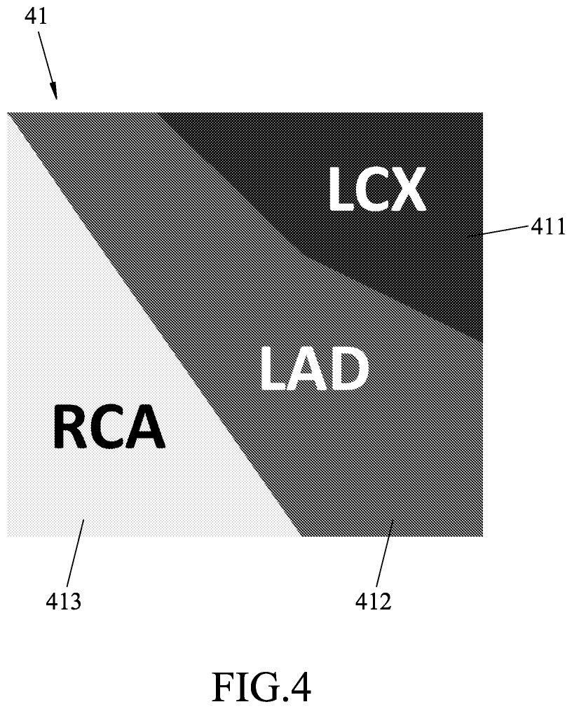

The storage unit 4 stores a comparison chart 41 (see FIG. 4) related to relative territories supplied by the three coronary arteries of a heart. Referring to FIG. 4, the comparison chart 41 indicates three comparison zones 411, 412, 413. The three comparison zones 411, 412, 413 from the top right corner to the bottom left corner of the comparison chart 41 respectively represent the left circumflex artery (LCX), the left anterior descending artery (LAD) and the right coronary artery (RCA). In some embodiments, the storage unit 4 may be non-volatile memory that is able to retain stored information even when the power is turned off, such as, but not limited to, flash memory, ferroelectric random-access memory, read-only memory, a hard disk drive (HDD) or a solid-state disk (SSD).

The processor 3 is configured to receive from the detector member 2 the ECG signals which have undergone the aforementioned amplification, filtering and conversion performed by the detector member 2. In some embodiments, the processor 3 determines, for each of the ECG signals, a duration of a QT interval and a duration of an RR interval of the ECG signal, wherein the QT interval is an interval from a start of the Q wave to an end of the T wave of the ECG signal, and the RR interval is an interval from a start of one QRS complex to a start of the next QRS complex of the ECG signal. The processor 3 calculates a plurality of characteristic values based on the durations of the QT intervals and the durations of the RR intervals of the ECG signals, wherein the characteristic values are dedicated to different characteristic locations on the chest of the body 1 within the detection area 100. The characteristic locations include the locations where the precordial electrodes 21 are placed.

Specifically, to calculate the characteristic values, the processor 3 first calculates durations of corrected QT (QTc) intervals of the ECG signals based on the durations of the QT intervals and the durations of the RR intervals of the ECG signals from the respective precordial electrodes 21, and then makes the durations of the QTc intervals serve as the plurality of characteristic values. Each of the durations of the QTc intervals may be calculated based on a formula of

$$QTc = \frac{QT}{\sqrt{RR}} ,$$

where QTc is a duration of a QTc interval, QT is a duration of a QT interval (unit: millisecond), and RR is a duration of an RR interval (unit: second).

However, in some embodiments, the processor 3 may make the durations of the QT intervals directly serve as the plurality of characteristic values. In other words, the durations of the QT intervals are not corrected by the durations of the RR intervals, and therefore the process of determining the durations of the RR intervals of the ECG signals may be omitted.

Moreover, in some embodiments, according to the number of the precordial electrodes 21 and different design needs, after calculating the durations of the QTc intervals, the processor 3 further calculates additional durations by using two-dimensional (2D) interpolation based on the durations of the QTc intervals and the locations of placement of the precordial electrodes 21 (for data augmentation), and makes the additional durations and the durations of the QTc intervals serve as the characteristic values. In this way, besides the locations of placement of the precordial electrodes 21, the characteristic locations corresponding to the characteristic values further include interpolated locations that respectively correspond to the additional durations calculated by using the 2D interpolation. The 2D interpolation may be, but not limited to, bilinear interpolation, 2D nearest-neighboring interpolation or bicubic interpolation.

In the embodiment where the number of the precordial electrodes 21 is sixteen, the processor 3 first determines for each of sixteen ECG signals, a duration of a QT interval and a duration of an RR interval of the ECG signal, then calculates sixteen durations of QTc intervals based on the durations of the QT intervals and the durations of the RR intervals, then calculates eight additional durations by using the 2D interpolation to obtain a total of twenty-four augmented durations of QTc intervals which include the sixteen durations of the QTc intervals and the eight durations, and finally makes the additional twenty-four augmented durations of the QTc intervals serve as the characteristic values.

The processor 3 is further configured to determine a smallest characteristic value among the plurality of characteristic values, and determine the characteristic location on the chest that corresponds to the smallest characteristic value as a location of chronic or acute myocardial ischemia in the body 1. Furthermore, the processor 3 compares the distribution of the characteristic values among the characteristic locations with the comparison chart 41 so as to determine a region of chronic or acute myocardial ischemia in the heart of the subject. The processor 3 is further configured to control, in response to receipt of an output command inputted via the input unit 5, the output unit 6 to output a detection result that indicates the location of myocardial ischemia in the body 1 and the region of myocardial ischemia in the heart.

The processor 3 is further configured to control the output unit 6 to output the characteristic values. Specifically, the output unit 6 is controlled to present the characteristic values in a color map, which indicates the characteristic values by using respective colors at positions of the color map corresponding to the respective characteristic locations, wherein the colors are used based on magnitudes of the respective characteristic values. The color map may be generated by the processor 3 by using a hypsometric coloring technique and/or a landform color shading technique in the art of cartography, that is, different colors, different tints of colors and/or different shades of colors are used to present different magnitudes of the characteristic values. In this way, a viewer is able to quickly perceive the distribution of the the characteristic characteristic values among locations with ease; for example, it would be relatively easy for a viewer to know where lower characteristic values are located by reading the color map. In this way, the viewer can determine the location of myocardial ischemia in the body 1 and the region of myocardial ischemia in the heart.

The processor 3 is further configured to, in response to receipt of a mode-selection command inputted via the input unit 5, operate in one of a first evaluation mode and a second evaluation mode based on the mode-selection command so as to determine an overall severity of myocardial ischemia of the subject. The processor 3 controls, in response to receipt of another output command, the output unit 6 to output an evaluation result indicating the overall severity thus determined.

In the first evaluation mode, the processor 3 calculates a dispersion parameter according to a parameter evaluation algorithm, which includes a formula of

$${SI}_{QTc} = \frac{1}{S} {\sum }_{k = 1}^{S} \{ ( \frac{1}{n} ) {\sum }_{i = 1}^{n} \vert {( QTc )}_{k} - {( QTc )}_{i} \vert \} ,$$

where SI_{QTc} is the dispersion parameter, S is a total number of the characteristic locations, (QTc)ₖ is a duration of the QTc interval corresponding to a specific characteristic location among the characteristic locations, n is a number of the characteristic locations closest to the specific characteristic location, and (QTc)ᵢ is a duration of the QTc interval corresponding to one of the characteristic locations closest to the specific characteristic location. The processor 3 then determines the overall severity based on the dispersion parameter thus calculated. In some embodiments, the greater the dispersion parameter, the greater the overall severity.

In the second evaluation mode, the processor 3 calculates a duration difference between a longest one and a shortest one among the durations of the QTc intervals, and determines the overall severity based on the duration difference thus calculated. In some embodiments, the greater the duration difference, the greater the overall severity. In some embodiments, the processor 3 may calculate a duration difference between a longest one and a shortest one among the augmented durations of QTc intervals.

The output unit 6 is configured to output information or data, e.g., the detection result, the color map and/or the evaluation result, under control of the processor 3. In some embodiments, the output unit 6 may include a display, a projector, a speaker, a printer, other suitable output devices, or combinations thereof.

Referring to FIGS. 2 and 3, the wearable unit 7 is to be worn by the subject, and more specifically on the body 1 of the subject. The precordial electrodes 21 are attached to the wearable unit 7 in advance, and when the wearable unit 7 is worn on the body 1, the precordial electrodes 21 would be placed on predetermined locations within the detection region 100 on the chest of the body 1 where measurement of electrical potentials for producing the ECG signals is desired. In some embodiments, the wearable unit 7 is a piece of clothing, such as a vest.

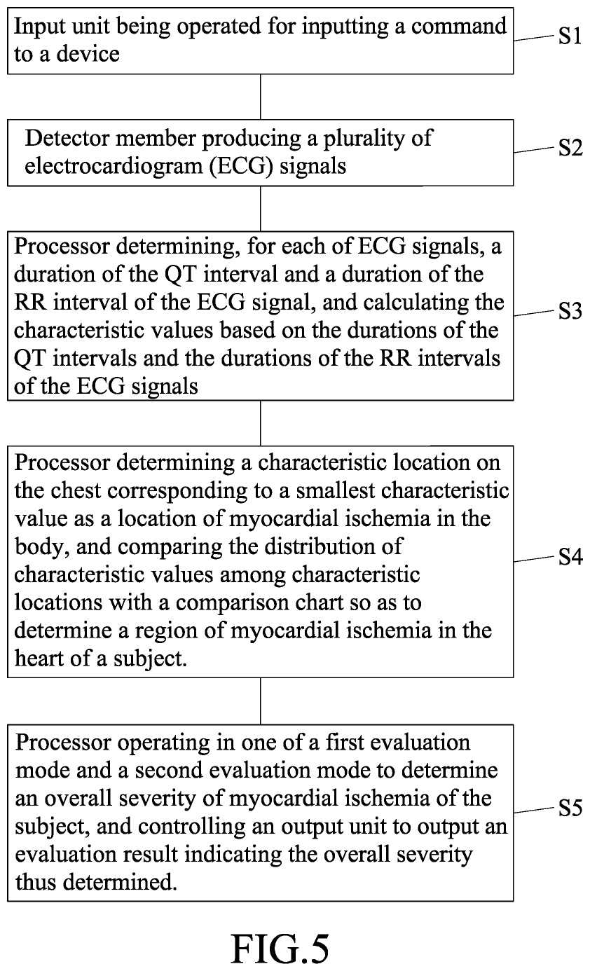

Referring to FIG. 5, an embodiment of a method for detecting myocardial ischemia according to the disclosure is to be implemented by using the system for determining cardiovascular characteristics exemplarily shown in FIGS. 1 and 2. The method includes steps S1 to S5.

In step S1, the input unit 5 is operated for inputting a command to the system. The command may be one of an activating command, an output command, a mode-selection command, and combinations thereof.

In step S2, the detector member 2 produces a plurality of ECG signals related to the body 1 of a subject. Specifically, the processor 3 controls, in response to receipt of the activating command, the electrodes 26, 21 to respectively measure electrical potentials at their respective locations of placement, and to cooperatively produce the ECG signals. At least some precordial electrodes 21 are placed on the left chest of the body 1.

In some embodiments, the ECG signals respectively correspond to the precordial electrodes 21, and thus respectively correspond to locations of placement of the precordial electrodes 21 (see FIG. 3).

In step S3, the processor 3 calculates a plurality of characteristic values. Specifically, the processor 3 receives the ECG signals from the detector member 2, and determines, for each of the ECG signals, a duration of the QT interval and a duration of the RR interval of the ECG signal. The processor 3 then calculates the characteristic values based on the durations of the QT intervals and the durations of the RR intervals of the ECG signals, wherein the characteristic values are dedicated to different characteristic locations on the chest of the body 1 within the detection area 100.

In some embodiments, the processor 3 first calculates durations of the QTc intervals of the ECG signals based on the durations of the QT intervals and the durations of the RR intervals of the ECG signals, and then makes the durations of the QTc intervals serve as the characteristic values.

In some embodiments, for data augmentation, the 2D interpolation is performed based on the durations of the QTc intervals and the locations of placement of the precordial electrodes 21 to obtain the augmented durations of QTc intervals which are made to serve as the characteristic values.

In step S4, the processor 3 determines a smallest characteristic value among the plurality of characteristic values, and determines the characteristic location on the chest corresponding to the smallest characteristic value as a location of myocardial ischemia in the body 1. Furthermore, the processor 3 compares the distribution of the characteristic values among the characteristic locations with the comparison chart 41 so as to determine a region of myocardial ischemia in the heart of the subject.

In some embodiments, the processor 3 further controls, in response to receipt of the output command, the output unit 6 to output a detection result that indicates the location of myocardial ischemia and the region of myocardial ischemia.

In some embodiments, the processor 3 further controls, in response to receipt of the output command, the output unit 6 to output the characteristic values. Specifically, the output unit 6 is controlled by the processor 3 to present the characteristic values in a color map mentioned above. In this way, a viewer is able to evaluate the distribution of the characteristic values among the characteristic locations with ease. In this way, by comparing the color map with the comparison chart 41, the viewer can determine exactly where myocardial ischemia has occurred.

In step S5, the processor 3, in response to receipt of the mode-selection command, operates in one of the first and second evaluation modes based on the mode-selection command so as to determine an overall severity of myocardial ischemia of the subject. The processor 3 controls, in response to receipt of another output command, the output unit 6 to output an evaluation result indicating the overall severity thus determined.

By these steps, the location in the body 1, the region of in the heart, and the overall severity of myocardial ischemia can be determined.

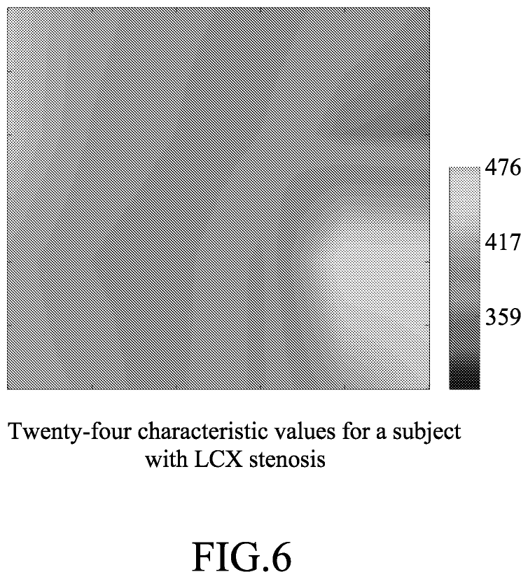

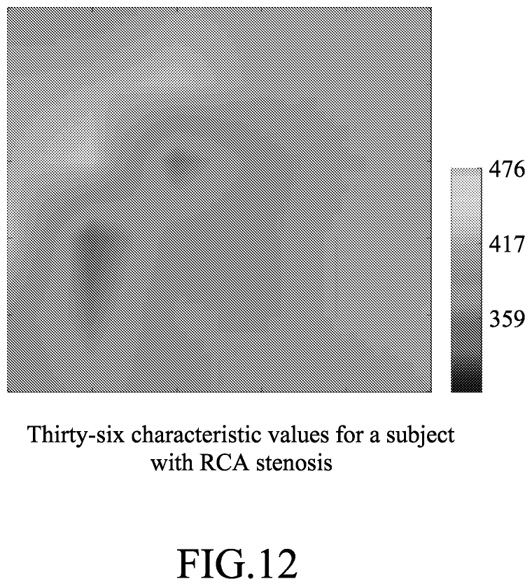

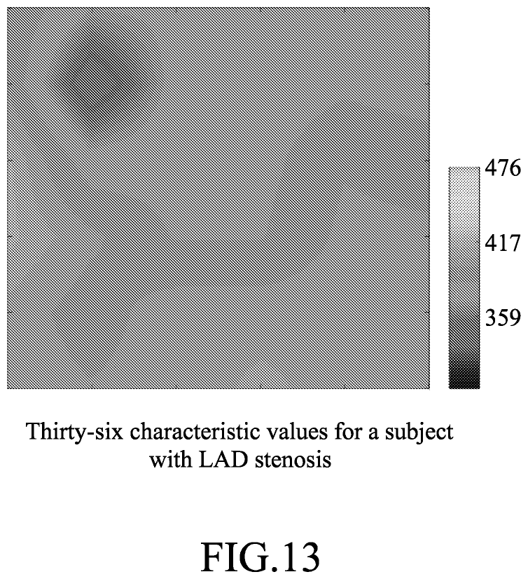

Referring to Table 1 below and FIGS. 4 and 6, a first exemplary case where the method according to this disclosure was performed on a subject who suffers from LCX stenosis is provided. Table 1 is a distribution table that presents the distribution of the characteristic values. In this case, sixteen precordial electrodes 21 were placed on the chest of the subject to obtain sixteen ECG signals; the processor 3 first calculated sixteen durations of QTc intervals of the sixteen ECG signals, next calculated eight additional durations by using the 2D interpolation for data augmentation to obtain a total of twenty-four augmented durations of QTc intervals, and finally made the twenty-four augmented durations of the QTc intervals serve as the characteristic values. Values presented in Table 1 are the twenty-four augmented durations, respectively, and are arranged based on the characteristic locations (i.e., the locations of placement of the sixteen precordial electrodes 21 and interpolated locations that respectively correspond to the eight additional durations). FIG. 6 is a color map displayed by the output unit 6 and indicating the characteristic values by using respective colors at positions of the color map corresponding to the respective characteristic locations, wherein the colors are used based on magnitudes of the characteristic values. It is noted that in FIG. 6 and FIGS. 7 to 9 and 11 to 14, the color maps are depicted as grayscale images, but in practice, these color maps may be color images.

TABLE 1

| | | | | | |
|---|---|---|---|---|---|
| 426 | 409 | 400 | 392 | 377 | 368 |
| 426 | 405 | 396 | 383 | 362 | 358 |
| 411 | 396 | 388 | 366 | 451 | 451 |
| 411 | 404 | 386 | 366 | 409 | 430 |

It is evident from Table 1 and FIG. 6 that the smallest characteristic value among the characteristic values is situated near the upper right corner in the table and the figure, and with reference to the comparison chart 41 in FIG. 4, it can be determined that a region of myocardial ischemia in the heart of the subject falls in the blood supply territory of LCX.

It is noted that the characteristic values may alternatively be calculated by using seventeen or eighteen precordial electrodes 21 to obtain ECG signals, and by obtaining augmented durations of QTc intervals using the 2D interpolation. In some embodiments, the characteristic values may be calculated based on ECG signals directly produced by twenty-four precordial electrodes 21 without using the 2D interpolation for data augmentation. In some embodiments, a number of the characteristic values presented in a distribution table is not limited to twenty-four. For example, a total of thirty-six characteristic values to be presented in the distribution table may correspond to thirty-six augmented durations of QTc intervals with twenty-four being obtained via twenty-four precordial electrodes 21 and twelve being obtained using the 2D interpolation.

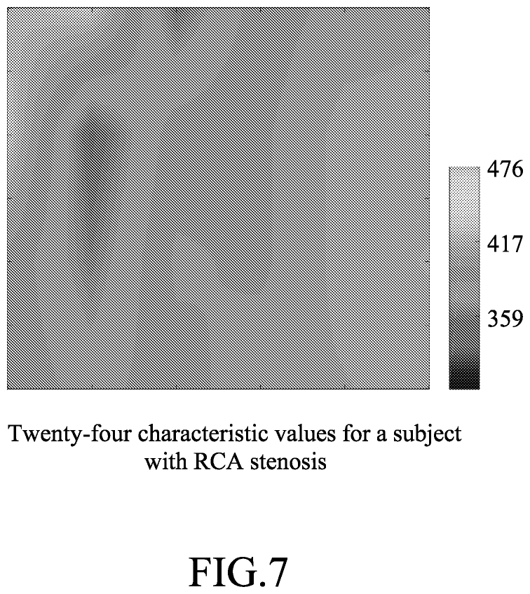

Referring to Table 2 below and FIGS. 4 and 7, a second exemplary case where the method according to this disclosure was performed on a subject who suffers from RCA stenosis is provided. Table 2 is a distribution table that presents the distribution of the characteristic values, which were obtained in the same manner as the first exemplary case. FIG. 7 is a color map displayed by the output unit 6 and indicating the characteristic values.

TABLE 2

| | | | | | |
|---|---|---|---|---|---|
| 417 | 428 | 356 | 390 | 400 | 404 |
| 428 | 348 | 390 | 394 | 409 | 409 |
| 394 | 360 | 398 | 394 | 409 | 413 |
| 394 | 377 | 392 | 402 | 405 | 409 |

It is evident from Table 2 and FIG. 7 that the smallest characteristic value is situated to the left in the table and the figure, and with reference to the comparison chart 41 in FIG. 4, it can be determined that a region of myocardial ischemia in the heart falls in the blood supply territory of RCA.

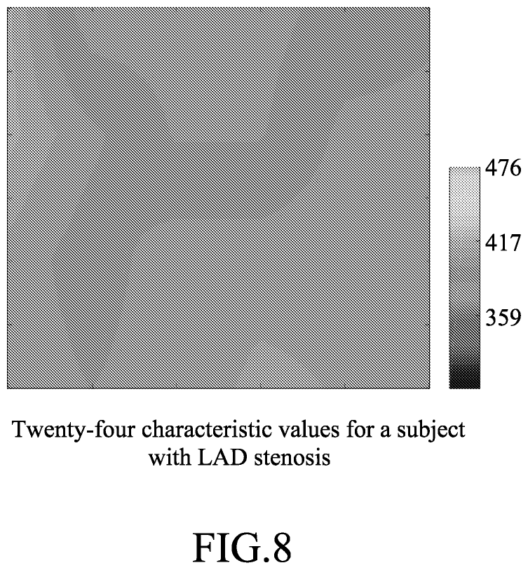

Referring to Table 3 below and FIGS. 4 and 8, a third exemplary case where the method according to this disclosure was performed on a subject who suffers from LAD stenosis is provided. Table 3 is a distribution table that presents the distribution of the characteristic values, which were obtained in the same manner as the first exemplary case. FIG. 8 is a color map displayed by the output unit 6 and indicating the characteristic values.

TABLE 3

| | | | | | |
|---|---|---|---|---|---|
| 415 | 374 | 378 | 378 | 388 | 393 |
| 415 | 399 | 382 | 382 | 399 | 399 |
| 403 | 390 | 403 | 403 | 403 | 399 |
| 403 | 397 | 402 | 407 | 405 | 402 |

It is evident from Table 3 and FIG. 8 that the smallest characteristic value is situated on the upper left-to-middle part of in the table and the figure, and with reference to the comparison chart 41 in FIG. 4, it can be determined that a region of myocardial ischemia in the heart of the subject falls in the blood supply territory of LAD.

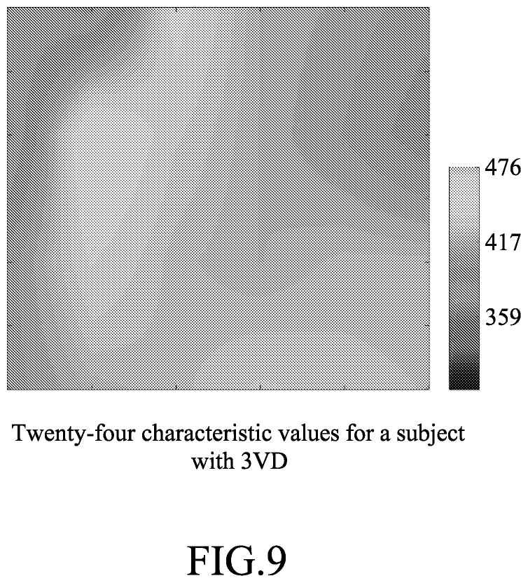

Referring to Table 4 below and FIGS. 4 and 9, a fourth exemplary case where the method according to this disclosure was performed on a subject who suffers from three-vessel disease (3VD) is provided. Table 3 is a distribution table that presents the distribution of the characteristic values, which were obtained in the same manner as the first exemplary case. FIG. 9 is a color map displayed by the output unit 6 and indicating the characteristic values.

TABLE 4

| | | | | | |
|---|---|---|---|---|---|
| 376 | 364 | 442 | 417 | 411 | 400 |
| 364 | 458 | 430 | 417 | 405 | 389 |
| 401 | 438 | 422 | 417 | 422 | 422 |
| 401 | 419 | 423 | 430 | 426 | 424 |

It is evident from Table 4 and FIG. 9 that the smallest characteristic value is situated near the upper left corner of the table and the figure, yet there are a couple of relatively small characteristic values dispersed in the upper left corner and the upper right corner. With reference to the comparison chart 41 in

FIG. 4, the upper left and upper right corners of the figure correspond to the three comparison zones 411, 412, 413, and it can thus be determined that a region of myocardial ischemia in the heart of the subject falls in the blood supply territories of three vessels (i.e., RCA, LCX, LAD).

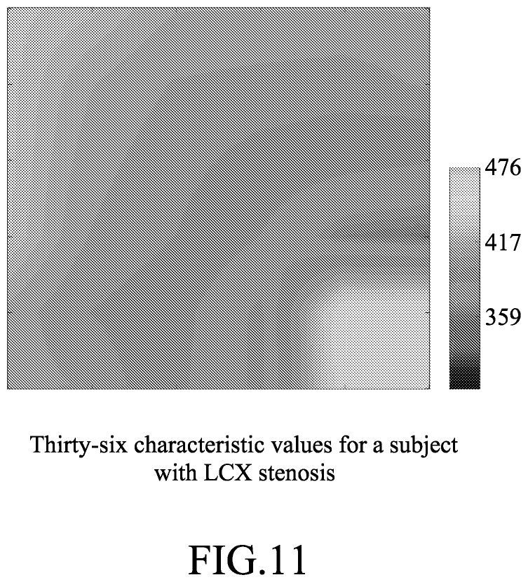

Tables 1 to 4 mentioned above and FIGS. 6 to 9 were obtained by using sixteen precordial electrodes 21. However, the method according to this disclosure is not limited to such implementation, and may be carried out by using a different number of precordial electrodes 21 to obtain a different number of ECG signals. Moreover, a different number of characteristic values may be calculated based on the ECG signals for subsequent detection and evaluation. For example, Tables 5 to 8 below respectively correspond to the aforementioned first to fourth exemplary cases, and FIGS. 11 to 14 also respectively correspond to the first to fourth exemplary cases, wherein, for each case, twenty-four precordial electrodes 21 (see FIG. 10) were used to obtain twenty-four ECG signals, from which twenty-four durations of QTc intervals were calculated, and twelve additional durations were calculated using the 2D interpolation, and the resulting thirty-six augmented durations of the QTc intervals served as the characteristic values. In this way, for each exemplary case, the characteristic values may be presented in a respective one of the distribution tables of Tables 5 to 8. Each of FIGS. 11 to 14 is a color map displayed by the output unit 6 and indicating the characteristic values by using respective colors.

TABLE 5

| | | | | | |
|---|---|---|---|---|---|
| 426 | 422 | 413 | 413 | 413 | 413 |
| 426 | 413 | 405 | 403 | 401 | 407 |
| 426 | 409 | 400 | 392 | 388 | 384 |
| 426 | 405 | 396 | 383 | 362 | 358 |
| 411 | 396 | 388 | 366 | 451 | 451 |
| 411 | 404 | 386 | 366 | 447 | 449 |

TABLE 6

| | | | | | |
|---|---|---|---|---|---|
| 398 | 398 | 411 | 411 | 411 | 411 |
| 413 | 417 | 425 | 409 | 408 | 410 |
| 417 | 428 | 356 | 390 | 405 | 408 |
| 428 | 348 | 390 | 394 | 409 | 409 |
| 394 | 360 | 398 | 394 | 409 | 413 |
| 394 | 377 | 392 | 402 | 398 | 405 |

TABLE 7

| | | | | | |
|---|---|---|---|---|---|
| 395 | 370 | 372 | 372 | 372 | 372 |
| 395 | 336 | 374 | 374 | 380 | 376 |
| 415 | 374 | 378 | 378 | 395 | 390 |
| 415 | 399 | 382 | 382 | 399 | 399 |
| 403 | 390 | 403 | 403 | 403 | 399 |
| 403 | 397 | 402 | 407 | 403 | 401 |

TABLE 8

| | | | | | |
|---|---|---|---|---|---|
| 413 | 422 | 391 | 391 | 391 | 391 |
| 385 | 372 | 360 | 389 | 394 | 392 |
| 376 | 364 | 442 | 417 | 401 | 394 |
| 364 | 458 | 430 | 417 | 405 | 389 |
| 401 | 438 | 422 | 417 | 422 | 422 |
| 401 | 419 | 424 | 430 | 430 | 426 |

Referring to FIG. 10, for the case of twenty-four precordial electrodes 21, from middle to side along the lateral direction of the body 1, four precordial electrodes 21 are placed at locations corresponding to the right edge 11 of the sternum, five at locations corresponding to a left edge 111 of the sternum, four at locations corresponding to a middle line 113 midway between the left edge 111 of the sternum and the left midclavicular line 112 of the body 1, four at locations corresponding to the left midclavicular line 112, four at locations corresponding to the left anterior axillary line 114 of the body 1, and three at locations corresponding to the left midaxillary line 13 of the body 1. From top to bottom along the inferior direction of the body 1, two of the twenty-four precordial electrodes 21 are placed at locations corresponding to the first intercostal space of the body 1, three at locations corresponding to the second intercostal space of the body 1, five at locations corresponding to the third intercostal space of the body 1, six at locations corresponding to the fourth intercostal space of the body 1, five at locations corresponding to the fifth intercostal space of the body 1, and three at locations corresponding to the sixth intercostal space of the body 1.

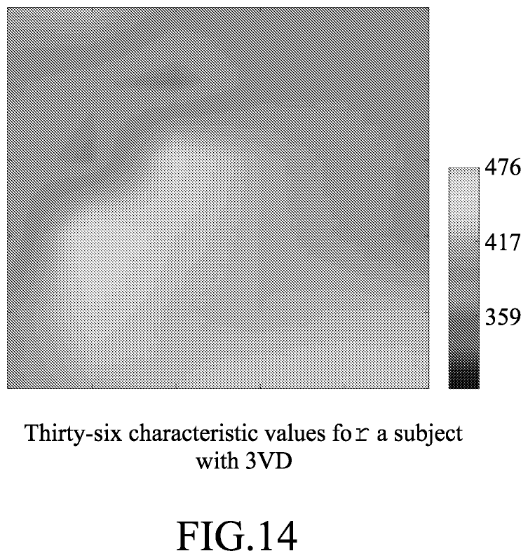

By comparing Tables 1 to 4 and FIGS. 6 to 9 with Tables 5 to 8 and FIGS. 11 to 14, it is found that for the same subject, no matter how many precordial electrodes 21 (e.g., sixteen in FIG. 3 or twenty-four in FIG. 10) are used to produce ECG signals for calculating the characteristic values (e.g., twenty-four or thirty-six characteristic values in the distribution tables), the same results can be obtained in terms of the location of myocardial ischemia in the body 1 and the region of myocardial ischemia in the heart. Moreover, in certain cases, when twenty-four precordial electrodes 21 are used to calculate thirty-six characteristic values for analysis, a position corresponding to a relatively small characteristic value can be more clearly observed. For example, in comparison with FIG. 9, FIG. 14 more clearly shows that the positions where the characteristic values are relatively small correspond to the three comparison zones 411, 412, 413 in the comparison chart 41.

On the other hand, when evaluating the overall severity of myocardial ischemia, no matter which of the first and second evaluation modes the processor 3 operates in, the main principle is to calculate a degree of dispersion of the characteristic values (e.g., a degree of dispersion of the augmented durations of the QTc intervals). The greater the dispersion parameter SI_{QTc} or the duration difference between a longest one and a shortest one among the augmented durations of the QTc intervals, the greater the overall severity of myocardial ischemia.

Taking the evaluation conducted by using sixteen precordial electrodes 21 as an example, for the first exemplary case based on Table 1, the dispersion parameter SI_{QTc} is 17.96 (unit: milliseconds hereinafter) and the duration difference between the longest one and the shortest one among the augmented durations of the QTc intervals is 93 (unit: milliseconds hereinafter), while for the third exemplary case based on Table 3, they are respectively 7.58 and 41. As a result, no matter which of the first and second evaluation modes is adopted, it is evident that the overall severity for the subject in the first exemplary case is greater than that for the subject in the third exemplary case. Since both the dispersion parameter SI_{QTc} and the duration difference for the subject in the first exemplary case indicate that the subject might need a more curative treatment compared to the subject in the third exemplary case, the first and second evaluation modes lead to the same evaluation result.

Moreover, taking the evaluation conducted by using twenty-four precordial electrodes 21 as an example, for the first exemplary case based on Table 5, the dispersion parameter SI_{QTc} is 13.35 and the duration difference between the longest one and the shortest one among the augmented durations of the QTc intervals is 93, while for the third exemplary case based on Table 7, they are respectively 9.11 and 79. As a result, it is also evident that the overall severity for the subject in the first exemplary case is greater than that for the subject in the third exemplary case. In other words, regardless of whether the analysis is conducted by using sixteen or twenty-four precordial electrodes 21, the same evaluation result is obtained. In the embodiment where twenty-four precordial electrodes 21 are used to collect ECG signals and twelve additional durations are calculated by using the 2D interpolation, when the dispersion parameter SI_{QTc} is greater than 9.4 or the duration difference is greater than 66, it means that the subject suffers from significant myocardial ischemia and may need a more curative treatment.

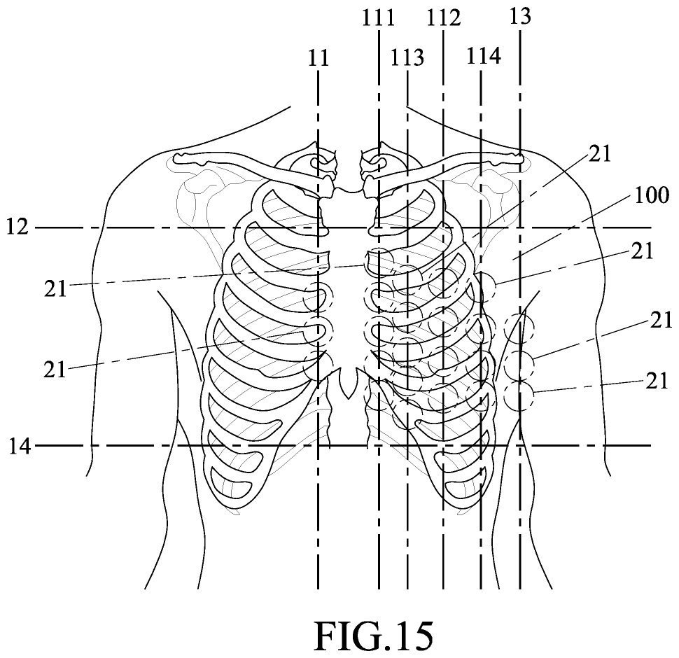

The arrangement of the precordial electrodes 21 is not limited to those shown in FIGS. 3 and 10. For example, referring to FIG. 15, another arrangement of twenty-four precordial electrodes 21 on the chest of a subject is illustrated. From middle to side along the lateral direction of a body 1, three of the twenty-four precordial electrodes 21 are placed at locations corresponding to the right edge 11 of the sternum, five at locations corresponding to a left edge 111 of the sternum, five at locations corresponding to a middle line 113 midway between the left edge 111 of the sternum and the left midclavicular line 112 of the body 1, four at locations corresponding to the left four midclavicular line 112, at locations corresponding to the left anterior axillary line 114 of the body 1, and three at locations corresponding to the left midaxillary line 13 of the body 1. From top to bottom along the inferior direction of the body 1, one of the twenty-four precordial electrodes 21 is placed at a location corresponding to the second intercostal space of the body 1, four at locations corresponding to the third intercostal space of the body 1, five at locations corresponding to the fourth intercostal space of the body 1, five at locations corresponding to the fifth intercostal space of the body 1, four at locations corresponding to the sixth intercostal space of the body 1. In addition, five of the twenty-four precordial electrodes 21 are placed at locations corresponding to the middle line 113 which is midway between the left edge 111 of the sternum and the left midclavicular line 112 of the body 1, and within a range from the third rib to the seventh rib of the body 1.

Based on experimentation and analysis, by adopting the arrangement of the twenty-four precordial electrodes 21 illustrated in FIG. 15, the same results in terms of the location of myocardial ischemia in the body, the region of myocardial ischemia in the heart and the overall severity can be obtained as those obtained by adopting the arrangements illustrated in FIGS. 3 and 10.

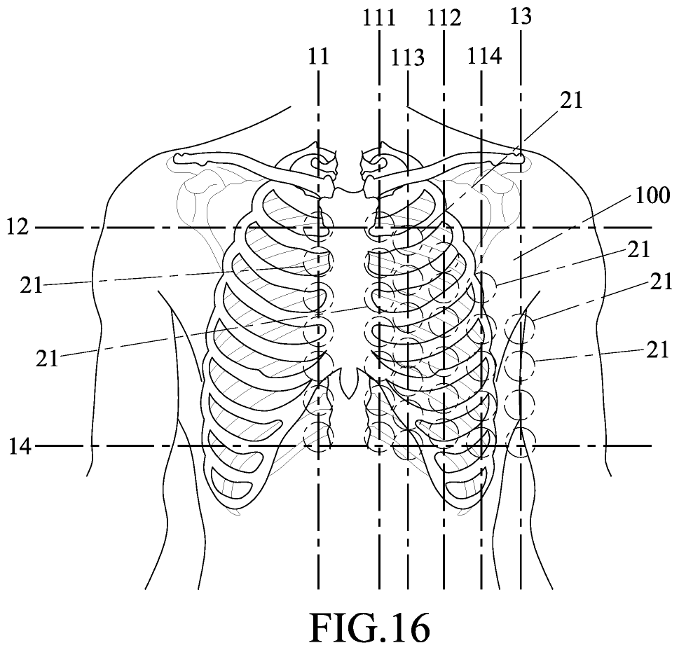

Alternatively, referring to FIG. 16, an exemplary arrangement of thirty-six precordial electrodes 21 on the chest of a subject is illustrated. From middle to side along the lateral direction of a body 1, seven of the thirty-six precordial electrodes 21 are placed at locations corresponding to the right edge 11 of the sternum, seven at locations corresponding to a left edge 111 of the sternum, seven at locations corresponding to a middle line 113 midway between the left edge 111 of the sternum and the left midclavicular line 112 of the body 1, six at locations corresponding to the left midclavicular line 112, five at locations corresponding to the left anterior axillary line 114 of the body 1, and four at locations corresponding to the left midaxillary line 13 of the body 1. From top to bottom along the inferior direction of the body 1, two of the thirty-six precordial electrodes 21 are placed at locations corresponding to the first intercostal space of the body 1, three at locations corresponding to the second intercostal space of the body 1, four at locations corresponding to the third intercostal space of the body 1, five at locations corresponding to the fourth intercostal space of the body 1, five at locations corresponding to the fifth intercostal space of the body 1, five at locations corresponding to the sixth intercostal space of the body 1, five at locations corresponding to the seventh intercostal space of the body 1, and seven at locations corresponding to the middle line 113 which is midway between the left edge 111 of the sternum and the left midclavicular line 112 of the body 1, and within a range from the second rib to the eighth rib of the body 1.

Based on experimentation and analysis, by adopting the arrangement of the thirty-six precordial electrodes 21 illustrated in FIG. 16, the same results in the aspects of the location, the region and the overall severity of myocardial ischemia can be obtained as those obtained by adopting the arrangements illustrated in FIGS. 3, 10 and 15.

To sum up, the system for determining cardiovascular characteristics according to this disclosure at least has the following advantages.
1. In this disclosure, the processor 3 calculates a plurality of characteristic values based on the durations of the QT intervals and the durations of the RR intervals of the ECG signals, and determines a location of myocardial ischemia in the body 1 based on the characteristic values. In comparison with the conventional approach where the evaluation is conducted based on amplitude variation in the waveform of ST segment, since the QT interval and the RR interval are less influenced by chest wall impedance, noise and baseline shift, evaluation error may be reduced.
2. In this disclosure, the processor 3 finds the smallest characteristic value, and determines the characteristic location on the chest corresponding to the smallest characteristic value as the location of myocardial ischemia. In comparison with the conventional approach where the evaluation is conducted based on waveforms reflecting ST segment elevation or depression in different grouped leads, the method of this disclosure may be performed with relative ease and may promote detection sensitivity.
3. The output unit 6 is controlled by the processor 3 to present the characteristic values in a color map, which indicates the characteristic values by using respective colors. A viewer is allowed to quickly perceive the distribution of the characteristic values among the characteristic locations with ease. In this way, the viewer can determine the location of myocardial ischemia in the body and the region of myocardial ischemia in the heart in a short span of time.
4. A single indicator, i.e., the dispersion parameter SI_{QTc} or the duration difference, is used to represent the overall severity of myocardial ischemia, which is a concise expression of the evaluation result.
5. By specific arrangement of at least sixteen precordial electrodes on the chest of a subject within the detection area 100, and by the processor 3 calculating the characteristic values, characteristics of ECG signals collected from the detection area 100 may be discovered thoroughly. Therefore, even if an inotrope is not used on the body 1, the location and the region related to chronic and acute myocardial ischemia of the subject can be determined. Moreover, since only sixteen precordial electrodes 21 at a minimum are needed to realize the method for detecting precordial ischemia, manufacturing cost of this device would not increase too much and the ease of use may be maintained.
6. By using the 2D interpolation for data augmentation, the sixteen durations of the QTc intervals calculated based on the ECG signals that are produced by the sixteen precordial electrodes 21 can be augmented to obtain twenty-four augmented durations of QTc intervals to serve as the characteristic values. Compared with conventional 12-lead electrocardiogram, the accuracy of judgment may be promoted for the method of this disclosure. In comparison with another conventional approach where more than a hundred electrodes are needed to obtain the ECG signals, the method of this disclosure decreases the number of electrodes required, so the cost and the labor to place the electrodes may be reduced.
7. By the design of the wearable unit 7, when the wearable unit 7 is worn on the body 1, the precordial electrodes 21 would be placed on the predetermined locations within the detection area 100 on the chest of the body 1 where measurement of electrical potentials for producing the ECG signals is desired. In this way, the procedure of positioning the precordial electrodes 21 on the body 1 may be simplified, the speed of positioning may be increased, and the correctness of positioning may be assured.

It is noted that even though the number of the precordial electrodes 21 may be sixteen, twenty-four, thirty-six, or other numbers, in some embodiments, the more the precordial electrodes 21, the more clearly a position corresponding to a relatively small characteristic value can be observed for analysis of myocardial ischemia. However, based on experimentation, when the number of the precordial electrodes 21 is thirty-six, the system of this disclosure is able to find out the exact location of myocardial ischemia in the body. Therefore, the number of the precordial electrodes 21 may be kept no greater than thirty-six so as to reduce the overall cost of the system.

In the description above, for the purposes of explanation, numerous specific details have been set forth in order to provide a thorough understanding of the embodiment (s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment (s), it is understood that this disclosure is not limited to the disclosed embodiment (s) but is intended to cover various arrangements included within the spirit and scope of the broadest interpretation so as to encompass all such modifications and equivalent arrangements.

1. A system for determining cardiovascular characteristics, the system to be disposed on the body of a subject, the body having a detection area which is defined by a right edge of the sternum, a horizontal line passing through the first intercostal space, the left midaxillary line, and a horizontal line passing through the eighth rib of the body, the system comprising:
a detector member which includes four limb electrodes to be placed on limbs of the subject, and at least sixteen precordial electrodes to be placed on the chest of the subject and spaced apart from each other, the limb electrodes and the precordial electrodes respectively measuring electrical potentials at locations of placement of the limb electrodes and the precordial electrodes, and cooperatively producing at least sixteen electrocardiogram (ECG) signals, each of the ECG signals including the P, Q, R, S and T waves, the precordial electrodes to be placed within the detection area in a manner that
at least two of the precordial electrodes are placed at locations corresponding to the right edge of the sternum, at least three of the precordial electrodes are placed at locations corresponding to a left edge of the sternum, at least three of the precordial electrodes are placed at locations corresponding to a middle line which is midway between the left edge of the sternum and the left midclavicular line of the body, at least four of the precordial electrodes are placed at locations corresponding to the left midclavicular line, at least two of the precordial electrodes are placed at locations corresponding to the left anterior axillary line of the body, and at least two of the precordial electrodes are placed at locations corresponding to the left midaxillary line of the body, and
at least three of the precordial electrodes are placed at locations corresponding to the third intercostal space of the body, at least five of the precordial electrodes are placed at locations corresponding to the fourth intercostal space of the body, at least four of the precordial electrodes are placed at locations corresponding to the fifth intercostal space of the body, at least one of the precordial electrodes is placed at a location corresponding to the sixth intercostal space of the body, and at least three of the precordial electrodes are placed at locations corresponding to the middle line which is midway between the left edge of the sternum and the left midclavicular line of the body, and within a range from the third intercostal space to the sixth rib of the body;
a processor in signal communication with the detector member for receiving the ECG signals, the processor being configured to calculate at least twenty-four characteristic values based on the ECG signals, wherein the at least twenty-four characteristic values are respectively dedicated to at least twenty-four characteristic locations on the chest of the subject within the detection area, the characteristic locations including the locations of placement of the precordial electrodes, the characteristic values serving as basis for determining a location of chronic or acute myocardial ischemia in the body and a region of chronic or acute myocardial ischemia in the heart of the subject; and
an output unit which is electrically connected to the processor and which is controllable by the processor to output the characteristic values;
wherein the processor is further configured to calculate durations of corrected QT (QTc) intervals of the ECG signals, and to make the durations of the QTc intervals serve as at least a part of the characteristic values;
wherein the processor is further configured to calculate a duration difference between a longest one and a shortest one among the durations of the QTc intervals, and to determine an overall severity of myocardial ischemia of the subject based on the duration difference thus calculated.

2.-5. (canceled)

6. The system as claimed in claim 1, wherein a number of the precordial electrodes of the detector member is smaller than or equal to thirty-six.

7. The system as claimed in claim 1, wherein a number of the precordial electrodes of the detector member is sixteen, the precordial electrodes placed within the detection area in a manner that
two of the precordial electrodes are placed at locations corresponding to the right edge of the sternum, three of the precordial electrodes are placed at locations corresponding to the left edge of the sternum, three of the precordial electrodes are placed at locations corresponding to the middle line that is midway between the left edge of the sternum and the left midclavicular line of the body, four of the precordial electrodes are placed at locations corresponding to the left midclavicular line, two of the precordial electrodes are placed at locations corresponding to the left anterior axillary line of the body, and two of the precordial electrodes are placed at locations corresponding to the left midaxillary line of the body, and
three of the precordial electrodes are placed at locations corresponding to the third intercostal space of the body, five of the precordial electrodes are placed at locations corresponding to the fourth intercostal space of the body, four of the precordial electrodes are placed at locations corresponding to the fifth intercostal space of the body, and one of the precordial electrodes are placed at locations corresponding to the sixth intercostal space of the body,
wherein three of the precordial electrodes placed at the locations each correspond to an intersection of the middle line with a respective one of the fourth rib, the fifth rib and the sixth rib of the body.

8. The system as claimed in claim 1, wherein a number of the precordial electrodes of the detector member is twenty-four.

9. The system as claimed in claim 8, wherein the precordial electrodes are placed within the detection area in a manner that:
four of the precordial electrodes are placed at locations corresponding to the right edge of the sternum, five of the precordial electrodes are placed at locations corresponding to the left edge of the sternum, four of the precordial electrodes are placed at locations corresponding to the middle line that is midway between the left edge of the sternum and the left midclavicular line of the body, four of the precordial electrodes are placed at locations corresponding to the left midclavicular line, four of the precordial electrodes are placed at locations corresponding to the left anterior axillary line of the body, and three of the precordial electrodes are placed at locations corresponding to the left midaxillary line of the body; and
two of the precordial electrodes are placed at locations corresponding to the first intercostal space of the body, three of the precordial electrodes are placed at locations corresponding to the second intercostal space of the body, five of the precordial electrodes are placed at locations corresponding to the third intercostal space of the body, six of the precordial electrodes are placed at locations corresponding to the fourth intercostal space of the body, five of the precordial electrodes are placed at locations corresponding to the fifth intercostal space of the body, and three of the precordial electrodes are placed at locations corresponding to the sixth intercostal space of the body.

10. The system as claimed in claim 8, wherein the precordial electrodes are placed within the detection area in a manner that:
three of the precordial electrodes are placed at locations corresponding to the right edge of the sternum, five of the precordial electrodes are placed at locations corresponding to the left edge of the sternum, five of the precordial electrodes are placed at locations corresponding to the middle line that is midway between the left edge of the sternum and the left midclavicular line of the body, four of the precordial electrodes are placed at locations corresponding to the left midclavicular line, four of the precordial electrodes are placed at locations corresponding to the left anterior axillary line of the body, and three of the precordial electrodes are placed at locations corresponding to the left midaxillary line of the body; and
one of the precordial electrodes is placed at a location corresponding to the second intercostal space of the body, four of the precordial electrodes are placed at locations corresponding to the third intercostal space of the body, five of the precordial electrodes are placed at locations corresponding to the fourth intercostal space of the body, five of the precordial electrodes are placed at locations corresponding to the fifth intercostal space of the body, four of the precordial electrodes are placed at locations corresponding to the sixth intercostal space of the body, and five of the precordial electrodes are placed at locations each correspond to an intersection of the middle line with a respective one of the third, the fourth, the fifth, the sixth and the seventh ribs of the body.

11. The system as claimed in claim 1, wherein a number of the precordial electrodes of the detector member is thirty-six, the precordial electrodes placed within the detection area in a manner that
seven of the precordial electrodes are placed at locations corresponding to the right edge of the sternum, seven of the precordial electrodes are placed at locations corresponding to the left edge of the sternum, seven of the precordial electrodes are placed at locations corresponding to the middle line that is midway between the left edge of the sternum and the left midclavicular line of the body, six of the precordial electrodes are placed at locations corresponding to the left midclavicular line, five of the precordial electrodes are placed at locations corresponding to the left anterior axillary line of the body, and four of the precordial electrodes are placed at locations corresponding to the left midaxillary line of the body; and
two of the precordial electrodes are placed at locations corresponding to the first intercostal space of the body, three of the precordial electrodes are placed at locations corresponding to the second intercostal space of the body, four of the precordial electrodes are placed at locations corresponding to the third intercostal space of the body, five of the precordial electrodes are placed at locations corresponding to the fourth intercostal space of the body, five of the precordial electrodes are placed at locations corresponding to the fifth intercostal space of the body, five of the precordial electrodes are placed at locations corresponding to the sixth intercostal space of the body, five of the precordial electrodes are placed at locations corresponding to the seventh intercostal space of the body, and seven of the precordial electrodes are placed at locations each correspond to an intersection of the middle line with a respective one of the second, the third, the fourth, the fifth, the sixth, the seventh and the eighth ribs of the body.

12. A system for determining cardiovascular characteristics, the system to be disposed on the body of a subject, the body having a detection area which is defined by a right edge of the sternum, a horizontal line passing through the first intercostal space, the left midaxillary line, and a horizontal line passing through the eighth rib of the body, the system comprising:
a detector member which includes four limb electrodes to be placed on limbs of the subject, and at least sixteen precordial electrodes to be placed on the chest of the subject and spaced apart from each other, the limb electrodes and the precordial electrodes respectively measuring electrical potentials at locations of placement of the limb electrodes and the precordial electrodes, and cooperatively producing at least sixteen electrocardiogram (ECG) signals, each of the ECG signals including the P, Q, R, S and T waves, the precordial electrodes to be placed within the detection area in a manner that
at least two of the precordial electrodes are placed at locations corresponding to the right edge of the sternum, at least three of the precordial electrodes are placed at locations corresponding to a left edge of the sternum, at least three of the precordial electrodes are placed at locations corresponding to a middle line which is midway between the left edge of the sternum and the left midclavicular line of the body, at least four of the precordial electrodes are placed at locations corresponding to the left midclavicular line, at least two of the precordial electrodes are placed at locations corresponding to the left anterior axillary line of the body, and at least two of the precordial electrodes are placed at locations corresponding to the left midaxillary line of the body, and
at least three of the precordial electrodes are placed at locations corresponding to the third intercostal space of the body, at least five of the precordial electrodes are placed at locations corresponding to the fourth intercostal space of the body, at least four of the precordial electrodes are placed at locations corresponding to the fifth intercostal space of the body, at least one of the precordial electrodes is placed at a location corresponding to the sixth intercostal space of the body, and at least three of the precordial electrodes are placed at locations corresponding to the middle line which is midway between the left edge of the sternum and the left midclavicular line of the body, and within a range from the third intercostal space to the sixth rib of the body;
a processor in signal communication with the detector member for receiving the ECG signals, the processor being configured to calculate at least twenty-four characteristic values based on the ECG signals, wherein the at least twenty-four characteristic values are respectively dedicated to at least twenty-four characteristic locations on the chest of the subject within the detection area, the characteristic locations including the locations of placement of the precordial electrodes, the characteristic values serving as basis for determining a location of chronic or acute myocardial ischemia in the body and a region of chronic or acute myocardial ischemia in the heart of the subject; and
an output unit which is electrically connected to the processor and which is controllable by the processor to output the characteristic values;
wherein the processor is further configured to
calculate durations of corrected QT (QTc) intervals of the ECG signals,
make the durations of the QTc intervals serve as at least a part of the characteristic values,
calculate a dispersion parameter based on the characteristic values according to a parameter evaluation algorithm, which includes a formula of
$${SI}_{QTc} = \frac{1}{S} {\sum }_{k = 1}^{S} \{ ( \frac{1}{n} ) {\sum }_{i = 1}^{n} \vert {( QTc )}_{k} - {( QTc )}_{i} \vert \} ,$$
where SI_{QTc} is the dispersion parameter, S is a total number of the characteristic locations, (QTc)ₖ is a duration of the QTc interval corresponding to a specific characteristic location among the characteristic locations, n is a number of the characteristic location(s) closest to the specific characteristic location, and (QTc)ᵢ is a duration of the QTc interval corresponding to one of the characteristic locations closest to the specific characteristic location, and
determine an overall severity of myocardial ischemia of the subject based on the dispersion parameter thus calculated.
